(19) United States
(12) Patent Application Publication
(10) Pub. No.: US 2025/0082200 A1
(43) Pub. Date: Mar. 13, 2025

(54) SLIT LAMP WITH BACKGROUND ILLUMINATOR

(71) Applicant: LUMENIS BE LTD., Yokneam (IL)

(72) Inventors: May Kleinman Ben Tsvi, Binyamina (IL); Adrian Graus, Kiriat Motzkin (IL); Yair Mandelstam-Manor, Yokneam (IL)

(21) Appl. No.: 18/830,057

(22) Filed: Sep. 10, 2024

Related U.S. Application Data
(60) Provisional application No. 63/537,643, filed on Sep. 11, 2023.

(51) Int. Cl. A61B 3/135

(52) U.S. Cl. CPC A61B 3/135

(57) ABSTRACT

A background illuminator for a slit lamp and method of providing background illumination for an eye. The background illuminator provides a background light beam comprising at least one of an infrared (IR) light beam, a visible light beam, and a combination of the IR light beam and the visible light beam. The background illuminator is configured to emit the background light beam on a human eye placed at a focal plane of interest of the slit lamp.

### RELATED APPLICATIONS

The present non-provisional application claims the priority benefit of U.S. provisional application No. 63/537,643, filed on Sep. 11, 2023, which is incorporated by reference herein in its entirety.

### FIELD OF THE INVENTION

The present disclosure relates to the field of ophthalmoscope devices, more particularly, but not exclusively, to illumination arrangements used in such devices.

### BACKGROUND

Ophthalmoscope devices including slit lamps are used to examine human eyes. Examining a human eye precisely and clearly depends on the quality of an image generated by an ophthalmoscope device. In a typical slit lamp, intense visible light is used to illuminate a target tissue in the human eye for viewing the eye. However, the use of intense visible light is limited due to eye glaring, which prevents the patient from keeping eyes open constantly during an examination and/or makes treatment painful.

A discussion of a slit lamp structure for an ophthalmoscope is found in U.S. Pat. No. 9,962,079 assigned to the assignee (LUMENIS LTD.,) of the present invention, the disclosure of which is incorporated herein by reference in its entirety.

The information disclosed in this background of the disclosure section is for enhancement of understanding of the general background of the invention and should not be taken as an acknowledgement or any form of suggestion that this information forms the prior art already known to a person skilled in the art.

### SUMMARY

The present disclosure relates to a background illumination for a slit lamp. In some embodiments, background illuminator(s) are added to the main illuminator or illumination module of a slit lamp, to reveal or show a surrounding background detail of an imaged object, e.g. a human eye, which the typical main illuminator of a slit lamp does not. The background illuminator(s) may aid a practitioner to better identify the object of interest and facilitate diagnosis and/or treatment decision making. In some embodiments, the background illuminator(s) are configured as an add-on module that can be added to a slit lamp. In some embodiments, the background illuminator(s) are integrated with the illumination module of the slit lamp device (e.g., the background illuminator(s) are designed with the illumination module into the slit lamp).

In some embodiments, the background illuminator(s) provide background illumination beam(s). The background illumination beam(s) are at least one of an infrared (IR) light beam, a visible light beam, and a combination of an IR light beam and a visible light beam. In modern ophthalmoscopes/slit lamps, a camera can be integrally or externally included to capture images and videos of the human eye at the focal plane of interest for diagnosis and/or treatment, in parallel to the image seen by the practitioner through the ophthalmoscope binoculars. IR light, even though not seen by the human eye, can be imaged by an IR-sensitive camera. In some embodiments, background illuminator(s) are externally mounted on an illuminating module of the slit lamp. The background illuminator(s) may be positioned to emit at least one of an IR light beam, a visible light beam, and a combination of an IR light beam and a visible light beam on an object placed at a focal plane of interest for the slit lamp or of a lens of the illuminating module.

In some embodiments, the background illuminator(s) includes one or more background illuminators for providing at least one of an infrared (IR) light beam, a visible light beam, and a combination of an IR light beam and a visible light beam and an associated light-directing element for each of the one or more background illuminators. In some embodiments, each of the one or more background illuminators and the associated light-directing element are arranged between a lens of an illuminating module of the slit lamp and an object placed at a focal length for a plane of interest of the slit lamp or of the lens of the illuminating module. In some embodiments, the background illuminators are configured to be aligned with the illumination module (or tower) at the same radius or optical path length while the illumination module is moved typically along an arc. The associated light-directing element reflects at least one of the IR light beam, the visible light beam, and the combination of the IR light beam and the visible light beam on the object.

In yet another embodiment, a method for enhancing an optical image of an eye using a slit lamp is disclosed. The method comprises providing a background illuminator that emits light from the background illuminator to project on an object placed at a focal plane of interest of a lens of an illuminating module of the slit lamp. The background illuminator may be externally mounted with respect to the illuminating module of the slit lamp.

In one embodiment, the background illuminator comprises an array of Light Emitting Diode (LEDs). In an embodiment, the array of LEDs is either a one-dimensional array or a two-dimensional array. In an embodiment, the array of LEDs is arranged either in a concentric circle or in a concentric square.

In an embodiment, the illuminating module of the slit lamp (which might include at least part of the background illuminator) comprises an internal light source or illuminator (e.g., located within an illumination tower of the slit lamp device) for providing at least one of an IR light beam, a visible light beam, and a combination of an IR light beam and a visible light beam. The illuminating module of the slit lamp can be configured as an array of LEDs, with various arrangements and shapes, similar to what is described above, where at least part of the LEDs forms the background illuminator.

In an embodiment, the background illuminator comprises an associated light-directing element for the background illuminator. In an embodiment, the associated light-directing element is an adjustable mirror and/or prism. The background illuminator allows adding an IR light beam in the surrounding of a visible light beam in a slit lamp. Imaging the IR-illuminated region with a camera sensitive to IR light thereby increases the signal to noise (SNR) of the image and yields an improved image quality of the background. This approach enhances diagnosis and/or treatment of the human eye.

Using IR illumination with improved SNR enables increased contrast of features in an image of the eye. Advantages include:
a) Enhancement of anatomical details of a human eye.
b) Improved image of retinal lesions/microaneurysms, leading to earlier detection of diabetic retinopathy.
c) Improved image for identification of Bruch's membrane rupture, leading to earlier detection of choroidal neovascularization (CNV).
d) Preventing inadvertent laser targeting of small blood vessels and the resulting risk of ocular bleeding.
e) Improved image of Meibomian Glands and improved diagnosis of Meibomian Gland Dysfunction (MGD).
f) Pupil dilation medication or drops not required when using only IR illumination.

In an aspect of the current disclosure there is a background illuminator for a slit lamp, the background illuminator comprising: a background illuminator for providing a background light beam comprising at least one of an infrared (IR) light beam, a visible light beam, and a combination of the IR light beam and the visible light beam, wherein the background illuminator is configured to emit the background light beam on a human eye placed at a focal plane of interest of the slit lamp. The background illuminator comprises an array of light emitting diodes (LEDs), the array of LEDs is a one-dimensional array, or the array of LEDs is a two-dimensional array. The two-dimensional array of LEDs is arranged in concentric shapes.

In another aspect, the background illuminator is integral with an illuminating module of the slit lamp, or externally mounted with respect to an illuminating module of the slit lamp. The background illuminator further comprising at least on light-directing element associated with the background illuminator, wherein the light-directing element is an adjustable mirror. And said light-directing element is positioned within an optical path of an illuminating module of the slit lamp and configured to reflect the background light beam and transmit light of the illuminating module.

In another aspect of the disclosure there is a set of background illuminators for a slit lamp, the background illuminators each comprising: one or more background illuminators for providing respective background light beams, each of said background light beams comprising at least one of an infrared (IR) light beam, a visible light beam, and a combination of the IR light beam and the visible light beam; and an associated light-directing element for each of the one or more background illuminators, wherein each of the one or more background illuminators and the associated light-directing element are configured to have the same optical distance to a human eye placed at a focal plane of interest of the slit lamp, and wherein the associated light-directing element reflects the background light beam on the human eye placed at the focal length of the illuminating module. The associated light-directing element is an adjustable mirror and each of the one or more background illuminators comprise an array of Light Emitting Diode (LEDs).

In yet another aspect there is the background illuminator of claim 13, wherein the array of LEDs is a one-dimensional array, the array of LEDs is a two-dimensional array, the two-dimensional array of LEDs is arranged in a concentric shape, or the background illuminator is integral with an illuminating module of a slit lamp.

In a further aspect of the disclosure there is a method for enhancing an optical image of an eye when using a slit lamp, the method comprising: providing a slit lamp, an illuminating module, and a background illuminator; and emitting from the background illuminator a background light beam on an object placed at a focal plane of interest of the slit lamp. The method, wherein the background illuminator is externally mounted with respect to the illuminating module and the slit lamp, the background illuminator comprises at least one of an Infrared (IR) light beam, a visible light beam, and a combination of the IR light beam and the visible light beam. The background illuminator comprises an array of light emitting diode (LEDs), the array of LEDs is either a one-dimensional array or a two-dimensional array, or wherein the array of LEDs is a two-dimensional array arranged in a concentric shape.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The accompanying drawings, which are incorporated in and constitute a part of this disclosure, illustrate exemplary embodiments, and together with the description, serve to explain the disclosed principles. In the drawings, the digit(s) of a reference number identifies the drawing in which the reference number first appears. The same numbers are used throughout the figures to reference like features and components. Some embodiments of systems and/or methods in accordance with embodiments of the present subject matter are now described below, by way of example only, and with reference to the accompanying figures.

FIG. 1A is a schematic diagram of a non-limiting exemplary environment for a background illuminator for a slit lamp in accordance with an embodiment of the present disclosure.

FIG. 1B is a schematic diagram a non-limiting exemplary environment for a background illuminator for a slit lamp in accordance with another embodiment of the present disclosure.

FIG. 1C is a schematic diagram of yet another non-limiting exemplary environment for a background illuminator for a slit lamp in accordance with another embodiment of the present disclosure.

FIGS. 2A, 2B, and 2C illustrate non-limiting arrangements of an array of light emitting diodes (LEDs) in accordance with some embodiments of the present disclosure.

FIG. 3 illustrates an image of a focal plane of interest of a human eye model using a background illuminator with a slit lamp in accordance with some embodiments of the present disclosure.

FIGS. 4A and 4B illustrate images of a human eye model, with and without use of a background illuminator with a slit lamp, respectively, in accordance with some embodiments of the present disclosure.

FIG. 5 illustrates a flowchart showing a method for enhancing an optical image of an eye using a slit lamp in accordance with some embodiments of the present disclosure.

FIG. 6 illustrates images of a human eye with IR background illumination for viewing a Meibomian Gland(s) in accordance with some embodiments of the present disclosure.

It should be appreciated by those skilled in the art that any block diagrams herein represent conceptual views of illustrative systems embodying the principles of the present subject matter. Similarly, it will be appreciated that any flowcharts, flow diagrams, state transition diagrams, pseudo code, and the like represent various processes which may be substantially represented in computer readable medium and executed by a computer or processor, whether or not such computer or processor is explicitly shown.

### DETAILED DESCRIPTION

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

While the disclosure is susceptible to various modifications and alternative forms, specific embodiment thereof has been shown by way of example in the drawings and will be described in detail below. It should be understood, however that it is not intended to limit the disclosure to the particular forms disclosed, but on the contrary, the disclosure is to cover all modifications, equivalents, and alternatives falling within the scope of the disclosure.

The terms “comprises”, “comprising”, or any other variations thereof, are intended to cover a non-exclusive inclusion, such that a setup, device or method that comprises a list of components or steps does not include only those components or steps but may include other components or steps not expressly listed or inherent to such setup or device or method. In other words, one or more elements in a system or apparatus proceeded by “comprises . . . a” does not, without more constraints, preclude the existence of other elements or additional elements in the system or method.

In the following detailed description of the embodiments of the disclosure, reference is made to the accompanying drawings that form a part hereof, and in which are shown by way of illustration specific embodiments in which the disclosure may be practiced. These embodiments are described in sufficient detail to enable those skilled in the art to practice the disclosure, and it is to be understood that other embodiments may be utilized and that changes may be made without departing from the scope of the present disclosure. The following description is, therefore, not to be taken in a limiting sense.

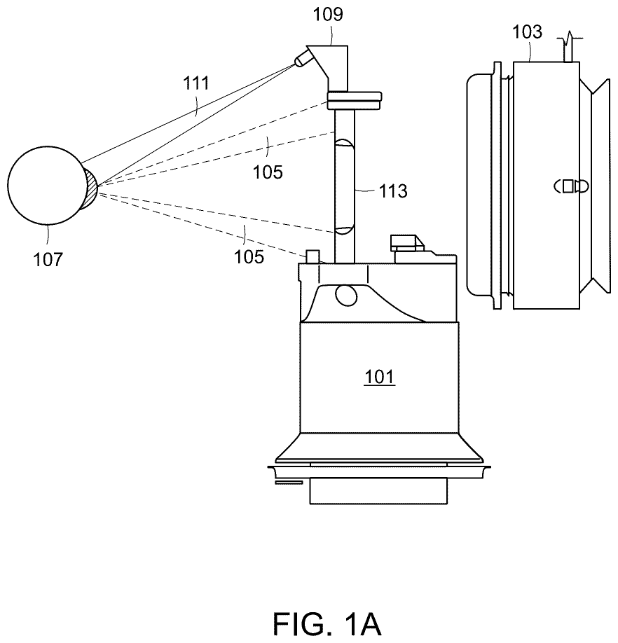

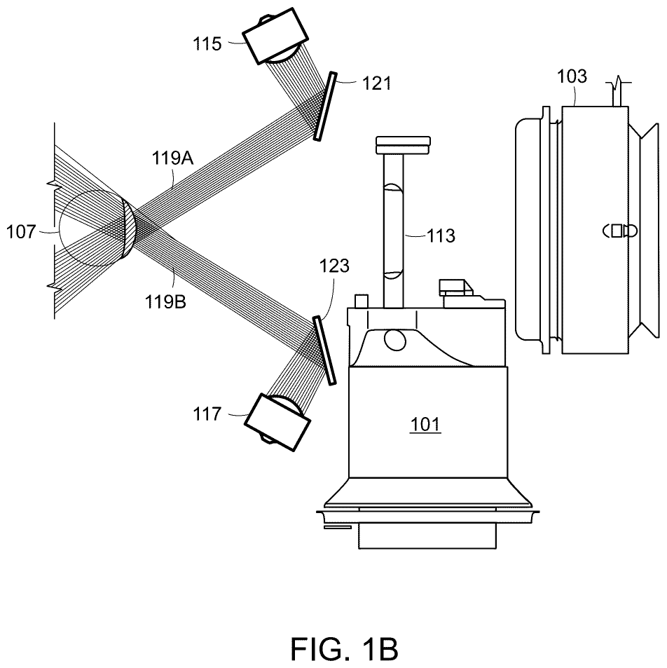

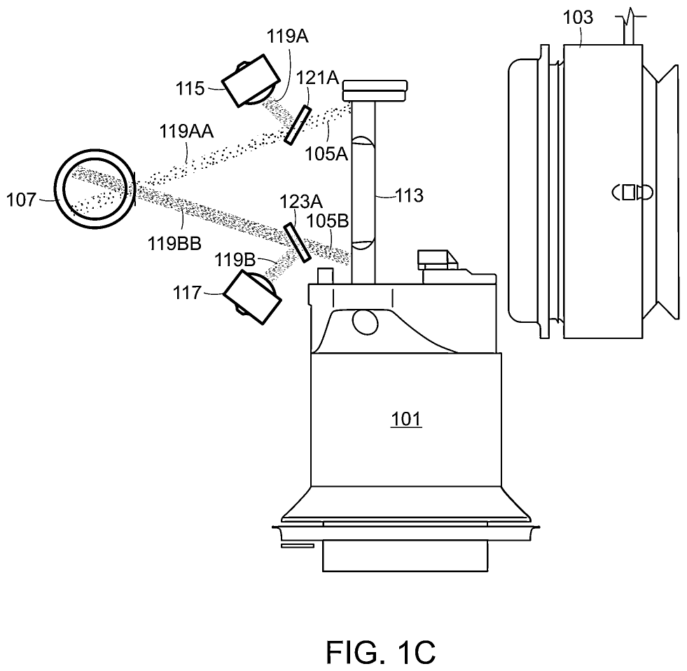

FIG. 1A illustrates a non-limiting exemplary configuration for a slit lamp with a background illuminator 109, in accordance with an embodiment of the present disclosure.

The slit lamp may include an illuminating module 101, a microscope/imaging module 103, and a lens or lenses (not shown in the figure) and mirror(s) 113 of the illuminating module 101. In some embodiments, the illuminating module 101 of the slit lamp comprises an internal light source (not shown in FIG. 1A) for providing at least one of an infrared (IR) light beam, a visible light beam, and a combination of an IR light beam and a visible light beam.

For ease of explanation, and by way of specific example, a visible light beam (or multiple beams) 105 emanating from the illuminating module 101 may be projected on the object 107 using the lenses (not shown in this figure) inside the illuminating module 101 and mirrors 113. The object 107 is placed at a focal plane of interest of the slit optical system 103. In some embodiments, the object 107 is a human eye.

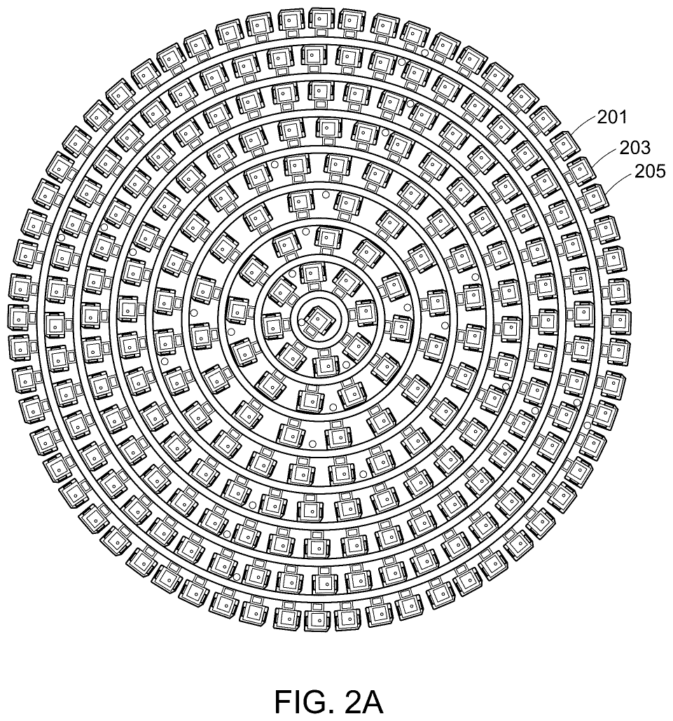

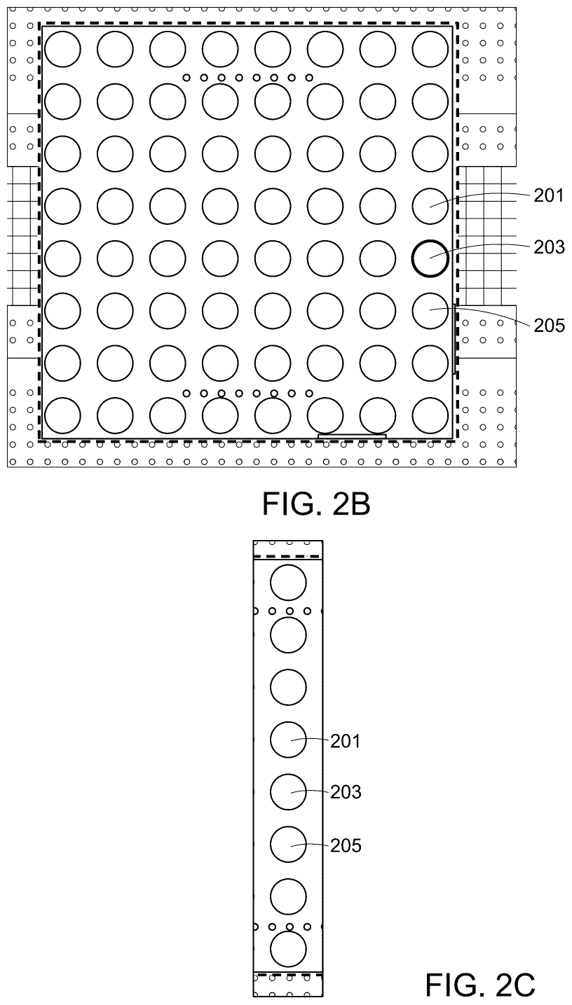

The background illuminator 109 may include an array of LEDs. (see FIGS. 2A-C) for providing at least one of an IR light beam, a visible light beam, and a combination of an IR light beam and a visible light beam. Reference 111 in FIG. 1A refers to at least one of an IR light beam, a visible light beam, and a combination of an IR light beam and a visible light beam originating from the background illuminator 109. In this example, the background illuminator 109 is externally mounted on the illuminating module 101 of the slit lamp. Further, the background illuminator 109 is positioned to emit at least one of the IR light beams, the visible light beam, and the combination of the IR light beam and the visible light beam on the human eye 107. Typically, the IR light beam is not visible by the human eye. In some embodiments, and when the illumination is only IR illumination, the pupil of an eye would not be affected and this eliminates the need for pharmacological pupil dilation (mydryasis) when viewing inner structures of the eye (e.g., the retina).

Optionally, the background illuminator 109 has an associated light-directing element (not present in the configuration of FIG. 1A). The associated light-directing element may be at least one of; mirror(s), adjustable mirror, prism(s), at least one lens, or any combination thereof. The associated light-directing element may direct the light beam 111 from the background illuminator 109 to the human eye 107.

In some embodiments, the background illuminator 109 comprises an array of light-emitting diodes (LEDs) 201, 203, 205, as shown in FIGS. 2A and 2B. In one embodiment, the array of LEDs 201, 203, 205 is arranged in concentric circles, as shown in FIG. 2A. In another embodiment, the array of LEDs 201, 203, 205 is arranged in a two-dimensional (2D) array. The array of LEDs 201, 203, 205 may be arranged in any shape, without limitation to the concentric circles or the concentric rectangles/squares.

In some embodiments, the array of LEDs 201, 203, 205 includes, but are not limited to, one or more LEDs at a center region of the concentric circles or the concentric squares for producing the visible light beam, surrounded by a plurality of LEDs in a peripheral region for producing the IR light beam. Alternatively, and in some embodiments, the regions of the visible and IR LEDs are reversed.

In some embodiments, the array of LEDs is a one-dimensional array as shown in FIG. 2C. In some embodiments, the array of LEDs is a two-dimensional array, such as in FIGS. 2A and 2B. In the specific example of the configuration of FIG. 1A, the array of LEDs is a one-dimensional array (also, referred as a linear array), as shown in FIG. 2C, of one or more LEDs, that is mounted above the illumination module 101 in a direction normal (typically, a vector perpendicular to the tangent plane of the surface at a point) to the optical axis of the lens 113. The one-dimensional array may be translatable and/or rotatable with respect to an X-axis and/or Y-axis, to give best overall illumination of the human eye 107. In some embodiments, the LEDs in the array are controlled individually or in groups to produce a desired illumination pattern based on the specific application and the illuminated human eye.

The visible light beam 105, coming from within the slit lamp, and the IR light beam 111, emanating from the array of LEDs in the background illuminator 109, may be projected on the human eye 107 and reflect specularly and/or diffusely from the human eye 107. The visible light beam 105 and the IR light beam 111 reflected from the human eye 107 may be detected with the imaging/microscope module 103 for examination. The microscope module 103 may include a focal-plane image sensor of the imaging module to capture the visible light beam 105 and the IR light beam 111 reflected from the human eye 107. The imaging module 103 may be sensitive to, but not limited to, all range of wavelengths of light used in the specific application, i.e. visible wavelength and IR wavelength (e.g., by selection of a focal-plane image sensor with a requisite spectral response). The imaging module 103 may be insensitive over some wavelength range(s). In some embodiments, the spectral sensitivity of the imaging module 103 is adjustable (e.g., by exchange of appropriate optical filters).

In one non-limiting embodiment, the background illuminator for providing a background beam comprising at least one of an IR light beam, a visible light beam, and a combination of an IR light beam and a visible light beam, is disposed in, and emanates from, the illuminating module 101 and the background beam is projected on the human eye 107. In this case, the background beam comprising the combination of the IR light beam and the visible light beam has a common optical path with the illuminating module 101.

FIG. 1B illustrates a non-limiting exemplary configuration for a slit lamp with background illuminators 115, 117, in accordance with another embodiment of the present disclosure.

The slit lamp may include an illuminating module 101, an imaging/microscope module 103, and mirrors 113 of the illuminating module 101; the illuminating module 101 may include one or more lenses (not shown). In some embodiments, the illuminating module 101 of the slit lamp comprises an internal illuminator (not shown in FIG. 1B) for providing at least one of an IR light beam, a visible light beam, and a combination of an IR light beam and a visible light beam.

In some embodiments, a visible light beam (not shown in FIG. 1B, similar to the visible light beam 105 in FIG. 1A) emanates from the illuminating module 101 and may be projected on the human eye 107 using the lenses inside the illuminating module 101 and mirrors 113. The human eye 107 may be placed at a focal plane of interest of the slit lamp system 103. In some embodiments, the human eye 107 may be a human eye.

The background illuminators 115, 117 may comprise an array of LEDs; see FIGS. 2A-C or any IR source for providing background light beams. Background light beams 119A and 119B may each be one of an IR light beam, a visible light beam, and a combination of an IR light beam and a visible light beam originating from the background illuminators 115 and 117.

In some embodiments, and by way of specific example, each of the one or more background illuminators 115, 117 has an associated light-directing/reflecting element(s), such as a mirror 121, 123. For instance, the background illuminator 115 may have an associated light directing element such as a mirror 121 and the background illuminator 117 may have an associated light directing element 123.

In some embodiments, as shown in the configuration of FIG. 1B, each of the one or more background illuminators 115, 117 and associated light directing elements 121, 123 are arranged between the mirror(s) 113 of the illuminating module 101 and the human eye 107 placed at the focal plane of interest of the slit lamp system 103. Further, each of the one or more background illuminators 115, 117 and the associated light directing element 121, 123 are arranged on either side of the focal length of the mirrors 113 of the illuminating module 101. The associated light directing elements 121, 123 reflect the background light beams 119A, 119B onto the human eye placed at the focal plane of interest of the illuminating module 101. In some embodiments, each of the associated light directing element 121, 123 is an adjustable or foldable mirror. In some embodiments, though not shown in FIG. 1B, the configuration includes more optical elements located in the optical paths of the light beam(s) from the illuminating module 101 and the background light beam(s) 119A, 119B. These optical elements are configured for coupling the light beams into a common optical path towards the human eye 107 and/or for emitting the light beams into regions of interest at the human eye 107. Each of the one or more background illuminators 115, 117 may comprise an array of LEDs, such as array of LEDs 201, 203, 205, as described above and as shown in FIGS. 2A and 2B. In one embodiment, the array of LEDs 201, 203, 205 is arranged in a concentric circle, as shown in FIG. 2A. In another embodiment, the array of LEDs 201, 203, 205 is arranged in a concentric rectangle/square, as shown in FIG. 2B. The array of LEDs 201, 203, 205 may be arranged in any shape without limitation to the concentric circle or the concentric rectangles/squares.

In some embodiments, the array of LEDs 201, 203, 205 includes, but not limited to, one or more LEDs at a center region of the concentric circle or the concentric rectangle/square for producing the visible light beam surrounded by a plurality of LEDs for producing the IR light beam in a peripheral region. Alternatively, and in some embodiments, the regions of the visible and IR LEDs are reversed.

In some embodiments, the array of LEDs is a one-dimensional array. In some embodiments, the array of LEDs is a two-dimensional array. In some embodiments, the LEDs in the array are controlled individually or in groups to produce a desired illumination scheme or pattern based on the specific application and the illuminated human eye.

For ease of description, and in some embodiments, the background light beams 119A and 119B in FIG. 1B are noted as IR light beams. IR light beam 119A from background illuminator 115 is reflected by associated light directing element 121 and projected on the human eye 107. Analogously, IR light beam 119B from background illuminator 117 may be reflected by associated light directing element 123 and projected on the human eye 107, as shown in FIG. 1B. The visible light beam (not shown in FIG. 1B) and the IR light beams 119A and 119B may be projected on the human eye 107 and may get reflected specularly and/or diffusely from the human eye 107. The visible light beam 105 and IR light beams 119A and 119B reflected from the human eye 107 is/are detected using the imaging/microscope module 103 for examination. In some embodiments, the imaging module 103 may include a camera sensor to capture the visible light beam 105 (see FIG. 1A) and IR light beams 119A and 119B reflected from the human eye 107. The imaging module 103 may be sensitive to, but not limited to, all wavelengths of light, i.e. visible wavelengths and IR wavelengths. The imaging module 103 may be insensitive over some wavelength range(s). In some embodiments, spectral sensitivity of the imaging module 103 is adjustable (e.g., by exchange of appropriate optical filters).

It is noted that although in this example two background illuminators 115 and 117, respective background light beams 119A, 119B and mirrors 121, 123 are described, it will be appreciated that only one or more than two background illuminators and respective background light beams and mirrors may also be employed.

Although working of background illuminators 115 and 117 of FIG. 1B is explained above with respect to IR light beams 119A and 119B as background light beams, in principle, working of background illuminators 115, 117 is similar for visible light beams, and a combination of an IR light beam and a visible light beam as background light beams 119A and 119B. Hence, the explanation for a visible light beam, and a combination of an IR light beam and a visible light beam as the background illuminators, while omitted here, does not and should not limit the scope of the invention.

In some embodiments, one or more of the background illuminators 115 and 117 for providing background beam(s) comprising at least one of an IR light beam, a visible light beam, and a combination of an IR light beam and a visible light beam, are disposed in, and emanate from, the illuminating module 101 and the background beams may be projected on the human eye 107. In this case, the background beam comprising the combination of the IR light beam and the visible light beam will have a common optical path with the illuminating module 101.

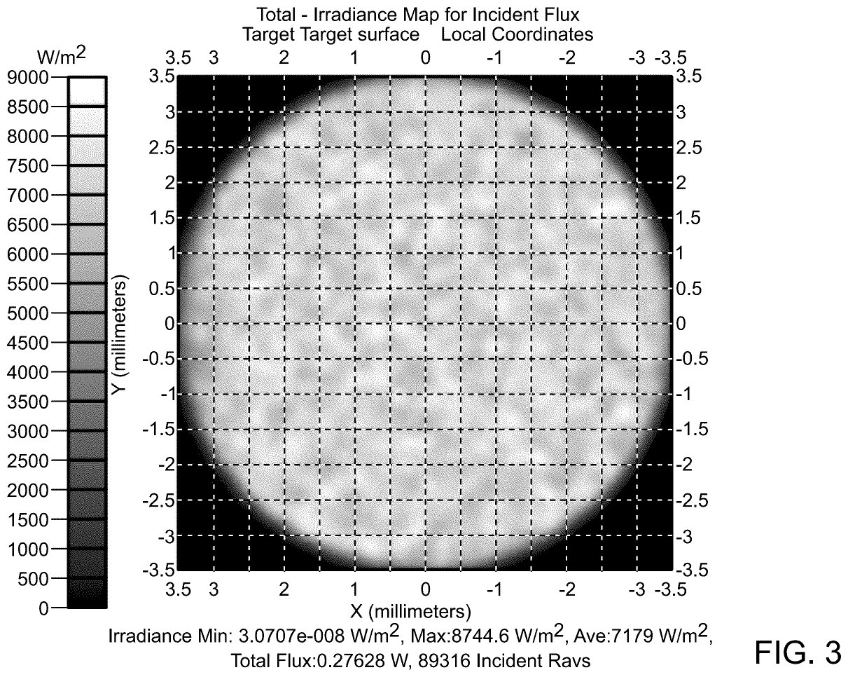

FIG. 3, and by way of specific example, illustrates an illumination image of a treatment or focal plane of interest of a human eye model. The image is produced by the imaging module 103 and illuminating module 101 (as in FIG. 1B) using background IR light beams, 119A, and 119B. The figure illustrates the illumination distribution on the treatment plane or focal plane of interest.

FIG. 1C illustrates another non-limiting exemplary configuration for a slit lamp with background illuminators 115, 117, in accordance with another embodiment of the present disclosure. The configuration is similar to that described in connection with FIG. 1B, except for the features described hereafter. In this example, two background illuminators 115 and 117 may produce background IR light beams 119A and 119B, respectively, which impinge on and are reflected by dichroic mirrors 121A, 123A respectively. In some embodiments, dichroic mirrors 121A and 123A are reflective to a first light spectrum, e.g. IR light, and transmissive (transparent) to a second light spectrum, e.g. visible light. These types of mirrors may be considered as beam combiners since they combine two light beams (reflected and transmitted) to the same optical path.

In some embodiments, and unlike light directing elements 121 and 123 shown in FIG. 1B, which are not located in the optical path of the light beams emanating from the illuminating module 101, dichroic mirrors 121A and 123A are positioned within the optical path of light beams 105A and 105B emanating from the illuminating module 101. As such, light beams 119A and 105A may be combined, after dichroic mirror 121A into a combined beam 119AA; and light beams 119B and 105B may be combined, after dichroic mirror 123A into a combined beam 119BB. Both combined beams 119AA and 119BB may be directed towards the human eye 107.

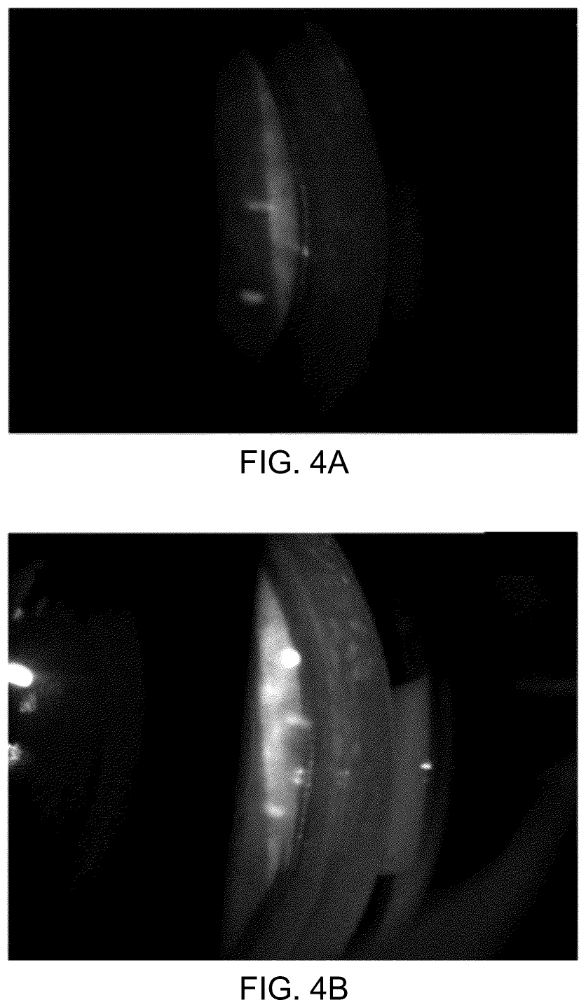

FIGS. 4A and 4B illustrate, by way of specific example, the importance of a background achieved by the systems and methods of the present invention to controllably achieve a desired illumination and visual inspection of specific portions of the human eye model. FIG. 4A illustrates an image of a human eye model without use of a background illuminator in the slit lamp, whereas FIG. 4B illustrates an image of the human eye model with the use of a background illuminator in the slit lamp. FIG. 4B clearly shows a better image quality of the trabecular meshwork in the human eye model as compared to the image in FIG. 4A, thereby aiding the practitioner in diagnosis and treatment decision-making. Moreover, using an IR background illumination does not add discomfort to the patient (visible illumination can be dazzling) while improving the SNR of the camera sensor which is sensitive to the IR spectral range.

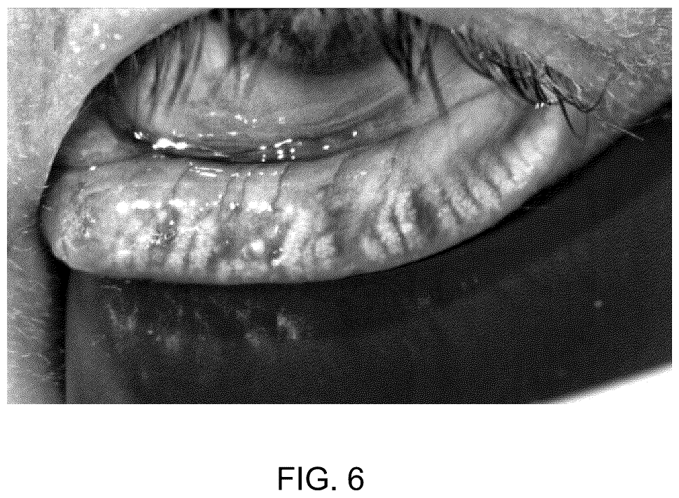

FIG. 6 illustrates, by way of specific example, the importance of a background achieved by the systems and methods of the present invention to controllably achieve a desired illumination and visual inspection of Meibomian Glands and therefore providing improved diagnosing of MGD.

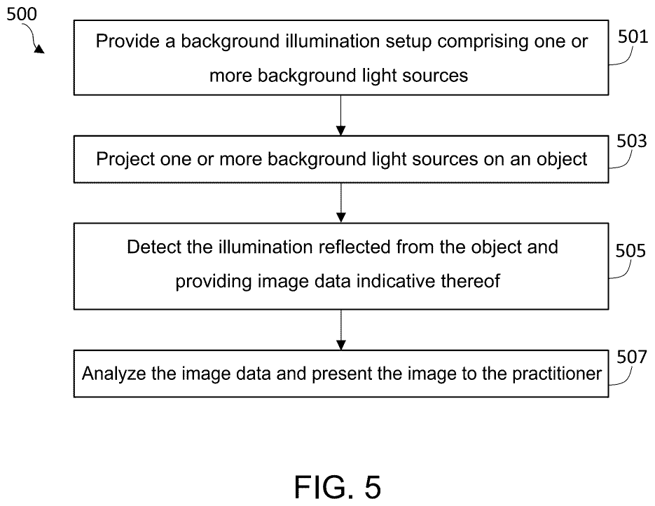

FIG. 5 illustrates a flowchart showing a method for enhancing an optical image of an eye using a slit lamp, in accordance with some embodiments of present disclosure. The method includes, inter alia, enhancing the contrast between different features, such as different biological structures within the eye, to help the practitioner in distinguishing and focusing on the desired features.

At step 501, the method includes providing background illumination with one or more background illuminators, in addition to the traditional illumination of the slit lamp device. As mentioned above, the background illuminator(s) may be either externally mounted with respect to the illuminating module of the slit lamp or internally integrated with the light source of the slit lamp, or both. The background illuminator emits at least one of an infrared (IR) light beam, a visible light beam, and a combination of an IR light beam and a visible light beam.

At 503, and in some embodiments, the method includes emitting background light beams from the background illuminator(s) on an human eye placed at a focal plane of interest of the slit lamp.

At 505, and in some embodiments, the method includes detecting the illumination reflected from the human eye and providing image data indicative thereof, the reflected illumination including both reflected light emanating from the slit lamp illumination and from the background illuminator.

At 507, and in some embodiments, the method includes analyzing the image data and presenting the image to the practitioner, enabling the practitioner to decide on a treatment plan.

Some of the advantages of the present disclosure are listed below:
a) The background illumination configurations allow addition of an IR light beam to a visible light beam in a slit lamp. Because the human eye is not sensitive to IR light, the background (IR) illuminator enables increasing overall illumination of a target tissue in the human eye. This approach enhances diagnosis and/or treatment of the human eye.
b) Increase in contrast due to the use of a background IR light beam enhances anatomical details of a human eye.
c) Increase in contrast due to the use of a background IR light beam in improves images of retinal lesions.
d) Increase in contrast due to the use of a background IR light beam improves images of microaneurysms, leading to earlier detection of diabetic retinopathy.
e) Increase in contrast due to the use of a background IR light beam improves identification of Bruch's membrane rupture, leading to earlier detection of choroidal neovascularization (CNV).
f) Increase in contrast due to the use of a background IR light beam improves identification of small blood vessels, enabling a practitioner to prevent inadvertent laser targeting of the small blood vessels, thereby reducing the risk of ocular bleeding.
g) Improved image of Meibomian Glands and improved diagnosis of Meibomian Gland Dysfunction (MGD).
h) Pupil dilation medication or drops not required when using only IR illumination.

The terms “an embodiment,” “embodiment,” “embodiments,” “the embodiment,” “the embodiments,” “one or more embodiments,” “some embodiments,” and “one embodiment” mean “one or more (but not all) embodiments of the invention(s),” unless expressly specified otherwise.

The terms “including,” “comprising,” “having,” and variations thereof mean “including but not limited to,” unless expressly specified otherwise.

The enumerated listing of items does not imply that any or all of the items are mutually exclusive, unless expressly specified otherwise.

The terms “a,” “an,” and “the” mean “one or more,” unless expressly specified otherwise.

A description of an embodiment with several components in communication with each other does not imply that all such components are required. On the contrary, a variety of optional components are described to illustrate the wide variety of possible embodiments of the invention.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article or a different number of devices/articles may be used instead of the shown number of devices or programs. The functionality and/or the features of a device may be alternatively embodied by one or more other devices which are not explicitly described as having such functionality/features.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims.

1. A background illuminator for a slit lamp, the background illuminator comprising:
a background illuminator for providing a background light beam comprising at least one of an infrared (IR) light beam, a visible light beam, and a combination of the IR light beam and the visible light beam,
wherein the background illuminator is configured to emit the background light beam on a human eye placed at a focal plane of interest of the slit lamp.

2. The background illuminator of claim 1, wherein the background illuminator comprises an array of light emitting diodes (LEDs).

3. The background illuminator of claim 2, wherein the array of LEDs is a one-dimensional array.

4. The background illuminator of claim 2, wherein the array of LEDs is a two-dimensional array.

5. The background illuminator of claim 4, wherein the two-dimensional array of LEDs is arranged in concentric shapes.

6. The background illuminator of claim 1, wherein the background illuminator is integral with an illuminating module of the slit lamp.

7. The background illuminator of claim 1, wherein the background illuminator is externally mounted with respect to an illuminating module of the slit lamp.

8. The background illuminator of claim 1, further comprising at least on light-directing element associated with the background illuminator.

9. The background illuminator of claim 8, wherein the light-directing element is an adjustable mirror.

10. The background illuminator of claim 8, wherein said light-directing element is positioned within an optical path of an illuminating module of the slit lamp and configured to reflect the background light beam and transmit light of the illuminating module.

11. A set of background illuminators for a slit lamp, the background illuminators each comprising:
one or more background illuminators for providing respective background light beams, each of said background light beams comprising at least one of an infrared (IR) light beam, a visible light beam, and a combination of the IR light beam and the visible light beam; and
an associated light-directing element for each of the one or more background illuminators,
wherein each of the one or more background illuminators and the associated light-directing element are configured to have the same optical distance to a human eye placed at a focal plane of interest of the slit lamp,
wherein the associated light-directing element reflects the background light beam on the human eye placed at the focal length of the illuminating module.

12. The background illuminator of claim 11, wherein the associated light-directing element is an adjustable mirror.

13. The background illuminator of claim 11, wherein each of the one or more background illuminators comprise an array of Light Emitting Diode (LEDs).

14. The background illuminator of claim 13, wherein the array of LEDs is a one-dimensional array.

15. The background illuminator of claim 13, wherein the array of LEDs is a two-dimensional array.

16. The background illuminator of claim 15, wherein the two-dimensional array of LEDs is arranged in a concentric shape.

17. The background illuminator of claim 11, wherein the background illuminator is integral with an illuminating module of a slit lamp.

18. A method for enhancing an optical image of an eye when using a slit lamp, the method comprising:
providing a slit lamp, an illuminating module, and a background illuminator; and
emitting from the background illuminator a background light beam on an object placed at a focal plane of interest of the slit lamp.

19. The method of claim 18, wherein the background illuminator is externally mounted with respect to the illuminating module and the slit lamp.

20. The method of claim 18, wherein the background illuminator comprises at least one of an Infrared (IR) light beam, a visible light beam, and a combination of the IR light beam and the visible light beam.

21. The method of claim 18, wherein the background illuminator comprises an array of light emitting diode (LEDs).

22. The method of claim 21, wherein the array of LEDs is either a one-dimensional array or a two-dimensional array.

23. The method of claim 21, wherein the array of LEDs is a two-dimensional array arranged in a concentric shape.
